(19) United States
(12) Patent Application Publication
(10) Pub. No.: US 2009/0028750 A1
(43) Pub. Date: Jan. 29, 2009

(54) Cleaning and Disinfection Swabbing Device for Needle-Free Intravenous (IV) Connectors

(76) Inventor: Dana Wm. Ryan, Nolensville, TN (US)

Correspondence Address: GORDON & JACOBSON, P.C., 60 LONG RIDGE ROAD, SUITE 407, STAMFORD, CT, 06902, US

(21) Appl. No.: 11/781,480

(22) Filed: Jul. 23, 2007

(51) Int. Cl. A61L 2/18

(52) U.S. Cl. 422/28; 422/292

(57) ABSTRACT

A device for cleaning and disinfecting needle-free intravenous connectors includes a cap, a sponge-like absorbent material impregnated with a disinfectant agent, and a hermetic seal. The cap defines a cavity whose walls are lined with the absorbent material and is sized to receive a needle-free intravenous connector. A hermetic seal provided for the cap seals the cavity and preserves the disinfectant agent contained in the absorbent material. The device is used by removing the hermetic seal, placing the cavity of the cap over the septum and thread profile of the connector, pushing the cap down on the septum portion of the IV connector so that the outer threads and septum of the connector slidingly interfere with the absorbent material containing the disinfectant agent, and twisting the cap back and forth to clean and disinfect the septum and thread profile. The device is discarded after use.

### CROSS-REFERENCE TO RELATED PATENTS

This application is related to U.S. Pat. No. 6,994,315, the complete disclosure of which is incorporated herein by reference.

### BACKGROUND OF THE INVENTION

1. Field of the Invention

This invention relates to a cleaning and disinfection swabbing device. More particularly, this invention relates to a device for cleaning and disinfecting the septum and threads on needle-free intravenous connectors.

2. State of the Art

Needle-free intravenous ports and connectors were introduced in the late 1980's to allow repeated infusions of fluids into a patient's bloodstream without the need to reinsert a needle into the patient's skin each time. The ports provided medical personnel a relatively simple means of introducing medicinal agents into the bloodstream without the repetitive physical and emotional traumas associated with needle injections. However, use of these ports has created contamination problems unforeseen at the time of their introduction.

Since widespread use of needle-free intravenous connectors began in the early 1990's, a significant increase in intraluminal catheter-related bloodstream infections has occurred. This increase in the number of bloodstream infections has resulted largely from contamination of the ports and/or connectors by airborne sources and also by direct handling of the components. It has been estimated that this type of bloodstream infection has resulted in excess of $11 billion in costs to healthcare facilities worldwide and a 12% to 25% mortality rate.

There is no widely accepted standardized procedure for cleaning and disinfecting the septum and threads on needle-free intravenous connectors. Currently, medical practitioners use methods that typically involve the application of a disinfectant prep pad to the connector's septum surfaces. A proper cleaning and disinfection procedure requires that both the septum and the threads of the needle-free intravenous connector be properly cleaned and disinfected by physical mechanical abrasion with adequate time for the antiseptic to kill microorganisms prior to accessing the needle-free I.V. connector. However, the current swabbing methods are inevitably inconsistent because medical practitioners may inadvertently miss areas or fail to clean and disinfect the septum and threaded areas of the I.V. connector effectively.

Those of ordinary skill in the art of intravenous medical device technology will appreciate that while even the best clinicians can make mistakes, the consequence of these mistakes is the inevitable downstream microorganism contamination and biofilm development of the internal fluid pathways of the needle-free IV connector and catheter walls leading to an increase in intraluminal catheter-related bloodstream infection rates.

### SUMMARY OF THE INVENTION

A device for cleaning and disinfecting needle-free IV connectors is provided and includes a cap, an absorbent material, a disinfectant agent, and a hermetic seal.

The cap of the present invention is cylindrical and has a closed end opposite an open end. The cap is preferably made of a polymer-based material and defines a cavity. A round flange also radially extends around the entrance to the cavity of the cap. The cavity of the cap is sized to be slightly larger than the outer diameter of a needle-free IV connector.

The absorbent material covers the interior surface area of the cap. The material is shaped to receive the proximal end of a needle-free IV connector. The absorbent material is preferably made of any substance that can be thinly form-fit to the interior wall of the cap within the cavity and maintain a disinfectant agent therein.

The disinfectant agent is any chemical compound that will effectively destroy contaminating microorganisms on the surface of an needle-free IV connector. The disinfectant agent of the present invention is preferably a liquid compound that is impregnated into the absorbent material during manufacture of the device. Most preferably, the disinfectant agent is isopropyl alcohol, iodophor, chlorhexidine gluconate, alcohol/chlorhexidine gluconate combination. or any other approved disinfectant agent.

A seal is also provided for the cap to hermetically close the cavity and preserve the disinfectant agent contained therein. The seal is preferably positioned across the round flange of the cap thereby covering the cavity. When sealed, contaminants and fluids are prevented from entering the cavity thereby preventing access to the interior of the device until use. The seal is removed when grasped at an edge of the round flange by a medical practitioner and pulled off from the cap.

After removal of the hermetic seal, the cap is placed over the proximal end of a IV connector. The cap is then pushed downward until it bottoms out and twisted back and forth (clockwise and counter-clockwise) over the connectors septum and threaded area so that the absorbent material containing the disinfectant agent interferes with the threads and the septum of the connector. This interference effectively cleans and disinfects the outer threads and septum of the IV connector simultaneously before the connector is accessed. The cleaning and disinfecting swabbing device and seal are then discarded after a single use.

Additional objects and advantages of the invention will become apparent to those skilled in the art of intravenous medical device design upon reference to the detailed description taken in conjunction with the provided figures.

### BRIEF DESCRIPTION OF THE DRAWINGS

FIG. 1 is a cross-sectional view of the sealed cleaning and disinfecting swabbing device for needle-free intravenous connectors;

FIG. 2 is a cross-sectional view of the cap;

FIG. 3 is a bottom view of the cap; and

FIG. 4 is a perspective view of a needle-free intravenous connector.

### DETAILED DESCRIPTION OF THE PREFERRED EMBODIMENTS

As used herein, the term “about” is to be understood in its broadest sense to mean +/−15% of a stated value.

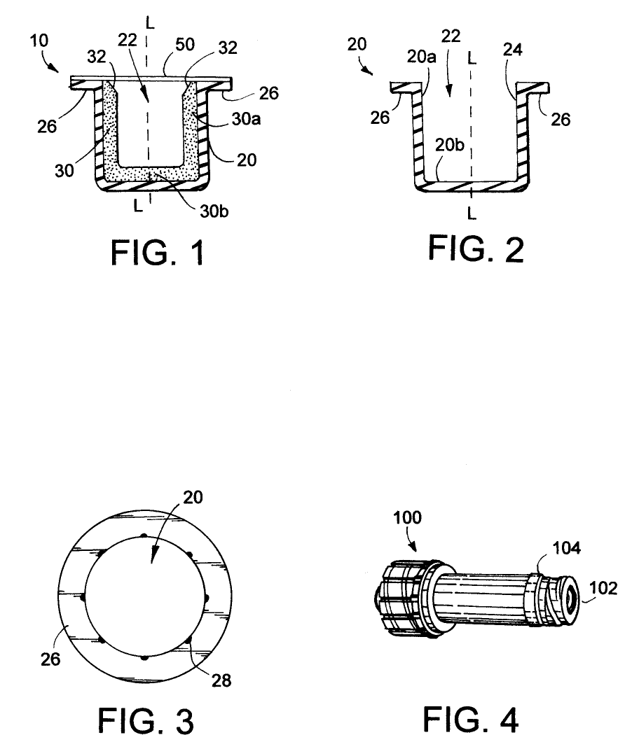

Turning now to FIGS. 1 and 2, a cleaning and disinfecting swabbing device 10 is shown having a cap 20. The cap 20 is substantially cylindrical in shape defining a longitudinal axis (L) and forming an opening end 20a, an opposite closed end 20b, and a tubular side wall 24. The cap 20 is preferably between about one-quarter and one-half inch in height along the longitudinal axis (L). The cap 20 may also be tapered in cross-section toward the closed end 20b. The cap 20 is preferably U-shaped in vertical cross-section and defines a cavity 22. The cavity 22 is sized for reception of a needle-free intravenous (IV) connector 100 (shown in FIG. 4). Needle-free IV connectors are described in detail in previously incorporated U.S. Pat. No. 6,994,315. The cavity 22 of the cap 20 is preferably dimensioned to have an inner diameter of about 0.394 inches and a height of about 0.45 inches. This inner diameter and height are slightly greater than the outer diameter and length requirements for female luer threads of medical connector devices as set by ANSI and ISO. This slight differences in diameters and heights (lengths) is designed to allow the cap 20 to fit closely around a connector 100 after the closed end 20b and side wall 24 are provided with an absorbent material 30, as discussed below.

As seen in FIGS. 1 and 2, a round flange 26 is also provided around the open end 20a of the cap 20 to provide a finger stop and seal seat. The round flange 26 extends radially outward from the longitudinal axis (L) of the cap 20 at the open end 20a of the cap 20. The round flange 26 preferably extends about 0.15 inches in radial length from the circumference of the open end of the cap 20. The round flange 26 helps prevent contamination by providing a physical barrier between the cavity 22 of the cap 20 and the medical practitioner.

The cap 20 and round flange 26 are preferably made of a rigid plastic or polymer-based material such as polyethylene, although other materials could be used to achieve a flexible, semi-rigid or thermoformed cap 20 into a desired configuration. Materials are appropriately selected for the cap 20 based on flexibility and surface characteristics because it is important that the medical practitioner can easily and tightly grasp the cap 20. Thermoformable materials that are resistant to bio-contaminants are optionally utilized in the formation of the cap 20. Alternatively, anti-bacterial or anti-microbial agents may be added into the selected material.

Turning now to FIG. 3, a bottom view of the cap 20 is shown wherein a plurality of vertical gripping ridges 28 project outwardly from the wall 24 in a preferred embodiment. The ridges 28 are preferably injection molded or thermoformed with the formation of the cap 20. The ridges 28 are semi-cylindrical in shape and preferably have a radius of about 0.01 inches. The ridges 28 extend in length about 0.4 inches from the underside of the round flange 26 down the outer side of the wall 24 such that each ridge 28 is parallel to the longitudinal axis (L) of the cap 20. The ridges 28 are provided to the cap 20 to assist the medical practitioner in obtaining a secure grasp on the device 10.

Referring back to FIG. 1, the absorbent material 30 is positioned in the cavity 22 on the closed end 20b and side wall 24 of the cap 20, and preferably covers the entire surface area of such locations. The absorbent material 30 preferably has a thickness of about 0.06 inches and therefore defines an inner diameter of about 0.275 inches (and an inner depth of about 0.39 inches) which is sized so that it will slidingly interfere with the outer threaded surfaces of an IV connector 100 (FIG. 4) when the cavity 22 of the cap 20 is pushed over the connector 100 thereby providing a tight fit. The absorbent material 30 is preferably tapered at 32 near the open end to facilitate insertion of an IV connector 100 (shown in FIG. 4). This tapering acts as a guide to introduce the cavity 22 of the cap over the IV connector. When the cap 20 is pushed over the IV connector 100, this sliding interference creates a tight fit wherein the absorbent material 30 contacts both the outer perimeter surface and/or threads 104 of the IV connector 100 at 30a and makes contact with the proximal septum 102 of the IV connector 100 at a bottom of the cavity 22 at 30b.

The absorbent material 30 is preferably made of a compressible foamed or sponge-like substance capable of holding a fluid or solution within. Examples of such substances include porous materials like cellulose as well as many polymer-based substances. Porous materials include a plurality of holes throughout the material for confinement of a liquid solution therein (e.g. a sponge). When the cavity 22 of the cap 20 is pushed over the proximal end of the IV connector 100 to contact the absorbent material 30, compression of the absorbent material causes a disinfectant agent to be released from the absorbent material 30 onto the surfaces of the IV connector 100.

The disinfectant agent is preferably maintained within the absorbent material 30 as previously described. The disinfectant agent is preferably impregnated into the absorbent material 30 just prior to the placement of a hermetic seal 50 over the cap 20. The disinfectant agent is preferably a liquid disinfecting compound or solution that readily destroys bio-contaminants and microorganisms associated with catheter-related bloodstream infections. Preferred disinfectant agents of the present invention include isopropyl alcohol, iodophor, chlorhexidine gluconate, alcohol/chlorhexidine gluconate combination. or any other approved disinfectant agent.

As previously stated, the disinfectant agents most preferably used in the present invention are isopropyl alcohol, iodophor, chlorhexidine gluconate, alcohol/chlorhexidine gluconate combination. or any other approved disinfectant agent. Chlorhexidine gluconate disinfects through disruption of microbial membranes and is commonly used in skin preps, antiseptic mouthwashes and other medical devices. Iodophors are mixtures of iodine with a solubilizing agent such as a surfactant. Iodine is a strong disinfecting compound that destroys microorganisms by oxidation of compounds in the cell membrane and cytoplasm. Free iodine however is highly reactive and is therefore included with solubilizing agents to improve useful shelf-life. Iodophors and iodine are particularly valuable as disinfecting agents because microorganisms are not known to develop resistant strains to the element. Isopropyl alcohol is a commonly used disinfectant agent available to the general public. Like iodophors, isopropyl alcohol destroys microorganisms by oxidation of cellular membranes. Of these three disinfectant agents, isopropyl alcohol is the most popular due to its disinfecting capabilities and its fast rate of evaporation. To prevent degradation of the disinfectant agents, the device 10 is equipped with a hermetic seal 50 to retain the disinfectant agent and to prevent contamination therein.

The hermetic seal 50 is included on the device 10 to prevent evaporation of the disinfectant agent as well as to prevent contamination of the device 10 prior to use. The hermetic seal 50 is preferably a thin disc of foil that seals the cavity 22 of the cap 20. Alternatively, the hermetic seal 50 is made of any material that prevents that transfer of fluids or contaminants through the seal 50 and provides airtight closure of the cavity 22. Depending on the material used, the hermetic seal 50 may include an antibacterial or antimicrobial agent as well. The hermetic seal 50 is affixed across the round flange 26 such that a medical practitioner can grasp an edge of the seal 50 to remove the hermetic seal 50 when the device 10 is to be used in cleaning and disinfecting an needle-free IV connector 100.

The device 10 is preferably used to disinfect the needle-free IV connector 100 momentarily before an IV set or a syringe (not shown) is attached to the IV connector. In use, the seal 50 is first removed by a medical practitioner. Next, the medical practitioner pushes the cavity 20 of the device 10 over the proximal end of a needle-free IV connector 100 such that the septum 102 of the connector 100 contacts the absorbent material 30 at the deepest point of the cavity 22. In this position, the absorbent material 30 of the device 10 simultaneously contacts both the septum 102 and the thread profile 104 of the IV connector 100. The threads 104 of the needle-free IV connector are typically external female luer threads. The absorbent material 30 is designed to flexibly accommodate and physically contact the entirety of the thread profile 104 and corresponding outer surface of the female luer so that a thorough cleaning and disinfection is achieved.

The contact between the IV connector 100 and the absorbent material 30 initiates the release of the disinfectant agent onto the IV connector 100 including the septum 102 and thread profile 104. Pushing the device 10 down until it bottoms out on the septum 102 and twisting back and forth (clockwise/counter clockwise) of the device 10 around the IV connector 100 causes increased contact with the absorbent material 30 and the disinfectant agent to insure complete cleaning and disinfection of the IV connector 100. Using this described technique, the cleaning and disinfection procedure of needle-free IV connectors could be standardized practice among medical practitioners to insure effective cleaning and disinfection of the septum 102 and thread profile 104 and reduce the probability of intraluminal catheter-related bloodstream infections.

Turning now to FIG. 4, a mating portion of the needle-free intravenous connector 100 is shown. The IV connector 100 includes the septum 102 at its proximal end and also the external thread profile 104. When coupled to vascular access catheter (not shown), the septum 102 provides a defensive barrier against downstream contamination into the IV connector 100 and subsequently the patient's vascular access catheter. The threads 104 are designed to mate with the male luer lock connector threads of a standard IV set, blood sampling device, or syringe for intravenous therapy or blood sampling and administration procedures.

Assembly of the device 10 is essentially a three phase process. In the first phase, the cap 20 is formed by injection molding or thermoforming. This formation phase may be completed by molding the wall 24, the flange 26, and the ridges 28 simultaneously in a single injection molding step wherein a liquefied polymer is injected into a mold of the fully formed cap 20. This liquefied polymer is then cooled and the mold removed to complete the formation of the cap 20. As described above, the inside diameter of the cap is precisely sized to allow the needle-free IV connector 100 to fit therein with an allowance for the thin absorbent material 30 to fit tightly therebetween.

The second phase is the introduction of the absorbent material 30 and the disinfectant agent into the formed cap 20. In a preferred method, the absorbent material 30 is preformed to the shape of the interior wall 24 of the cap 20. The preformed absorbent material 30 is then inserted by bonding or heat staking into the cap 20. Once positioned, the absorbent material 30 is impregnated with a chosen disinfectant.

The third and final phase of the assembly of the cleaning and disinfection swabbing device 10 is the sealing of the cavity 22 to preserve the disinfectant agent. Several means are available to achieve a hermetic seal 50 across the flange 26. In a preferred method, a thin layer of tin foil having a polymer coating is placed onto the flange 26 such that the foil covers the entirety of the cavity 22. The foil covered cavity is then passed through an electromagnetic field wherein the foil is heated. The heated foil causes the melting of the polymer. The melted polymer coating overflows from the heated foil and bonds to the exterior of the flange 26. This bonding creates the hermetic (airtight) seal 50 necessary to preserve the disinfectant agent. The foil piece may optionally include an extended tab which may be grasped by the user of the device 10 for ease of removal of the seal 50. Other means of hermetic sealing will be apparent to one of ordinary skill in the art of packaging engineering design.

There have been described and illustrated herein embodiments of a cleaning and disinfection swabbing device for needle-free intravenous connectors. While particular embodiments of the invention have been described, it is not intended that the invention be limited thereto, as it is intended that the invention be as broad in scope as the art will allow and that the specification be read likewise. Thus, while a particularly shaped cleaning and disinfection swabbing device for needle-free IV connectors has been shown and described, it will be appreciated that the exterior of the device may have a different shape without affecting function, e.g., conical in longitudinal section and/or hexagonal in cross-section. Similarly, the device as disclosed could have different internal dimensions provided that a sliding interference fit is obtained and the device is of sufficient length to effectively clean and disinfect the IV connector including its septum and thread profile. Furthermore, many disinfectant agents used to destroy microorganisms other than those specifically disclosed may be used in the present invention. It will therefore be appreciated by those skilled in the art that yet other modifications could be made to the provided invention without deviating from its scope as claimed.

What is claimed is:

1. A cleaning and disinfection swabbing device for a needle-free intravenous connector having a septum area and external threads, said swabbing device consisting essentially of:
a) a cap having a U-shaped cross-section, a side wall, an end wall, and defining a cavity;
b) an absorbent material affixed in said cap on the side wall and the end wall and defining an inner diameter of about 0.275 inches, wherein said absorbent material is shaped for simultaneous contact with the external thread profile and the septum of the standard needle-free intravenous connector;
c) a disinfectant agent in said absorbent material; and
d) a removable hermetic seal covering said cavity of said cap.

2. The device of claim 1, wherein:
said cap comprises a polymeric material.

3. The device of claim 1, wherein:
said cap includes at least one external gripping ridge.

4. The device of claim 1, wherein:
said cap includes a round flange around said cavity.

5. The device of claim 4, wherein:
said removable hermetic seal is affixed to said round flange.

6. The device of claim 1, wherein:
said disinfectant agent includes isopropyl alcohol.

7. The device of claim 1, wherein:
said disinfectant agent includes iodophor.

8. The device of claim 1, wherein:
said disinfectant agent includes chlorhexidine gluconate.

9. The device of claim 1, wherein:
said absorbent material includes cellulose.

10. The device of claim 1, wherein:
said absorbent material defines an interior depth of about 0.39 inches.

11. A cleaning and disinfection swabbing device for a standard needle-free intravenous connector having a septum area and external threads, said swabbing device consisting essentially of:
a) a cap having a U-shaped cross-section, a side wall, an end wall, and defining a cavity having a diameter of about 0.394 inches and a height of about 0.45 inches;
b) an absorbent material affixed in said cap on the side wall and the end wall and thereby defining an inner diameter of about 0.275 inches an interior depth of about 0.39 inches, wherein said absorbent material is shaped for simultaneous contact with the external thread profile and the septum of the standard needle-free intravenous connector;
c) a disinfectant agent in said absorbent material; and
d) a removable hermetic seal covering said cavity of said cap.

12. The device of claim 11, wherein:
said cap comprises a polymeric material.

13. The device of claim 11, wherein:
said cap includes at least one external gripping ridge.

14. The device of claim 11, wherein:
said cap includes a round flange around said cavity.

15. The device of claim 14, wherein:
said removable hermetic seal is affixed to said round flange.

16. The device of claim 11, wherein:
said disinfectant agent includes isopropyl alcohol.

17. The device of claim 11, wherein:
said disinfectant agent includes iodophor.

18. The device of claim 11, wherein:
said disinfectant agent includes chlorhexidine gluconate.

19. The device of claim 11, wherein:
said absorbent material includes cellulose.

20. A method of cleaning a needle-free intravenous connector having external threads extending along a length from the proximal end thereof and having a septum at the proximal end thereof, comprising the steps of:
a) providing a cleaning and disinfection swabbing device consisting essentially of,
i) a cap having a U-shaped cross-section, a side wall, an end wall, and defining a cavity,
ii) an absorbent material affixed in said cap on the side wall and the end wall thereby defining an inner diameter of about 0.275 inches wherein said absorbent material is configured for simultaneous contact with the thread profile and the septum of a standard needle-free intravenous connector,
iii) a disinfectant agent provided to said absorbent material, and
iv) a removable hermetic seal covering said cavity of said cap;
b) removing said hermetic seal covering from said cavity of said cap;
c) placing said cavity of said cap over an needle-free intravenous connector such that said absorbent material contacts the septum and the external thread profile of the intravenous connector; and
d) rotating said cap back and forth about the needle-free intravenous connector such that the septum and the external thread profile of the intravenous connector are cleaned and disinfected by the disinfectant agent provided to said absorbent material.

21. A method according to claim 20, further comprising:
e) disposing of said swabbing device.

22. The method of claim 20, wherein:
said disinfectant agent includes one of isopropyl alcohol, iodophor, chlorhexidine gluconate, chlorhexidine gluconate/isopropyl alcohol or any regulatory approved disinfectant agent.

23. The method of claim 20, wherein:
said absorbent material includes cellulose.
